(19) United States
(12) Patent Application Publication
(10) Pub. No.: US 2015/0196468 A1
(43) Pub. Date: Jul. 16, 2015

(54) URETHANE FOAM IMPREGNATED WITH COSMETIC COMPOSITION

(71) Applicant: AMOREPACIFIC CORPORATION, Seoul (KR)

(72) Inventors: Kyung Nam Kim, Yongin-si (KR); Jung Sun Choi, Yongin-si (KR); Kyung Ho Choi, Yongin-si (KR); Yeong Jin Choi, Yongin-si (KR); Ha Jin Jung, Yongin-si (KR)

(21) Appl. No.: 14/430,009

(22) PCT Filed: Sep. 4, 2013
(86) PCT No.: PCT/KR2013/007985

§ 371 Date: Mar. 20, 2015

(30) Foreign Application Priority Data
Sep. 21, 2012 (KR) 10-2012-0105209
Sep. 3, 2013 (KR) 10-2013-0105296

(51) Int. Cl. A61K 8/02; C08G 18/48; C08G 18/08; C08J 9/40

(52) U.S. Cl. CPC A61K 8/0216; C08J 9/40; C08G 18/48; C08G 18/14; C08J 2375/08; C08J 2205/046; C08G 2101/00

(57) ABSTRACT

The present invention relates to a dry polyether-based urethane foam impregnated with a cosmetic composition.

### TECHNICAL FIELD

The present disclosure relates to a dry polyether-based urethane foam impregnated with a cosmetic composition.

### BACKGROUND ART

Cosmetic compositions have frequently been used indoors in the past. However, users have used cosmetic compositions outdoors more and more in accordance with a change in life style including generalization of leisure activities. Therefore, cosmetic compositions convenient to use and carry have been increasingly on demand.

To enhance convenience in use and portability of cosmetic compositions, sponges made of various kinds of materials have been impregnated with cosmetic compositions. However, it is not easy to find a material excellent in durability, filling ability, impregnating ability and discharging ability. Therefore, there has been a need for developing a material suitable for impregnation with a cosmetic composition.

### REFERENCES OF THE RELATED ART

(Patent Document 1) Korean Patent Publication No. 10-2009-0100643

### DISCLOSURE

### Technical Problem

A technical problem to be solved by the present disclosure is to provide a dry polyether-based urethane foam suitable for impregnation with a cosmetic composition.

### Technical Solution

In one general aspect, there is provided a dry polyether-based urethane foam impregnated with a cosmetic composition.

In another general aspect, there is provided a cosmetic comprising a dry polyether-based urethane foam impregnated with a cosmetic composition.

### Advantageous Effects

According to the embodiments of the present disclosure, the dry polyether-based urethane foam has excellent durability, filling ability and discharging ability when it is impregnated with a cosmetic composition, and thus is suitable for impregnation with a cosmetic composition.

### DESCRIPTION OF DRAWINGS

FIG. 1 is a graph illustrating the filling ability of a dry polyether-based urethane foam and that of a wet polyether-based urethane foam as a function of hardness.

FIG. 2 is a graph illustrating the discharging ability of a dry polyether-based urethane foam and that of a wet polyether-based urethane foam as a function of hardness.

FIG. 3 is a photographic view of a cosmetic including a dry polyether-based urethane foam impregnated with a cosmetic composition according to an embodiment.

FIG. 4 is a schematic view illustrating a process of forming a reticulated structure of a dry polyether-based urethane foam according to an embodiment.

### BEST MODE

In one aspect, there is provided a dry polyether-based urethane foam impregnated with a cosmetic composition. In another aspect, the dry polyether-based urethane foam may function as a carrier on which a cosmetic composition is supported and retained.

As used herein, “urethane foam” means a foamed and solidified polyurethane resin, and may also be expressed as “foamed urethane”.

As used herein, “durability” means the degree of a urethane foam to maintain as it is without melting, tearing or swelling when it is impregnated with a cosmetic composition and stored at a predetermined temperature for a predetermined time. As used herein, “filling ability” means the ability of a urethane foam to fill a cosmetic composition therein, and may be represented as time required for filling a predetermined amount of cosmetic composition. As used herein, “discharging ability” means an amount of cosmetic composition discharged when taking the cosmetic composition from a urethane foam impregnated therewith. When taking the cosmetic composition, the cosmetic composition is required to be discharged in an adequate amount, not too much, but not too little.

Urethane foams may be classified into a polyester-based urethane foam and a polyether-based urethane foam. A polyether-based urethane foam is more resistant against humidity as compared to a polyester-based urethane foam, and thus is not broken easily under a high-humidity condition and shows high stability. In addition, a polyether-based urethane foam has a larger cellular structure as compared to a polyester-based urethane foam, and thus has improved air permeability, cushiony feel, softness and flexibility. Further, a polyether-based urethane foam requires reduced cost for production, and thus has higher cost-efficiency as compared to a polyester-based urethane foam.

According to an aspect of the present disclosure, the dry polyether-based urethane foam is for use in impregnation with a cosmetic composition, and has a constitution different from the constitution of the conventional cosmetic applicator (also called ‘puff’ or ‘cosmetic sponge’). In particular, ‘cosmetic applicator’ serves to apply a cosmetic composition to the user's skin upon use. In other words, a cosmetic applicator is intended to transfer a cosmetic agent to the skin by allowing the applicator itself to be coated with a cosmetic agent and to be in contact with the user's skin, and thus contains no cosmetic composition in itself. On the contrary, the material for impregnation with a cosmetic agent according to the present disclosure is impregnated with a cosmetic agent and retains the cosmetic agent in itself before use (i.e., requires durability and filling ability). Then, another cosmetic applicator is to be coated with an adequate amount of cosmetic composition upon use (i.e., requires discharging ability). That is, the material for impregnation with a cosmetic agent according to the present disclosure is not in direct contact with the skin but merely is impregnated with a cosmetic agent, and thus requires the use of another applicator. Therefore, ‘cosmetic applicator’, i.e., ‘puff’ does not allow easy application of a cosmetic composition onto the user's skin upon use, when the cosmetic composition infiltrates into the deep internal portion of sponge. Accordingly, it is preferred that a cosmetic agent hardly infiltrates into the deep internal portion of sponge. On the contrary, the foam impregnated with a cosmetic composition according to the present disclosure is a material for impregnation with a cosmetic agent and requires its impregnation with a cosmetic agent as an essential function. As described above, the foam according to the present disclosure is clearly different from the conventional cosmetic applicator (puff).

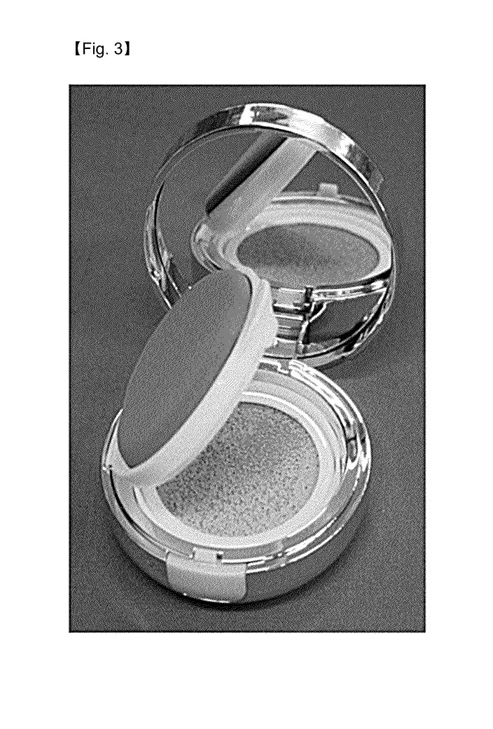

FIG. 3 shows a product including the material impregnated with a cosmetic composition according to the present disclosure. It can be seen that the product may further include a separate cosmetic applicator in addition to the material for impregnation with a cosmetic composition.

Polyether-based urethane foams may be classified into dry polyether-based urethane foams and wet polyether-based urethane foams depending on manufacturing processes. Dry sponges are obtained by introducing foam blocks to a gas chamber and injecting a gas thereto to carry out explosion and cell-opening. Thus, such a dry process requires high precision because the cell structure and hardness of the resultant product depend on the gas injecting amount. Wet sponges are obtained by introducing blocks into a container filled with a liquid phase (chemical) and carrying out cell-opening by a chemical action. Such a wet process is used mainly for producing soft foam products, and is expensive due to the use of different liquid phases.

A dry polyether-based urethane foam generally has a larger pore size as compared to a wet polyether-based urethane foam. While a wet polyether-based urethane foam is foamed by a wet process and shows hydrophilic surface property, a dry polyether-based urethane foam shows oleophilic surface property. A dry polyether-based urethane foam is commercially available as EZ93N (trademark). As compared to a wet polyether-based urethane foam, a dry polyether-based urethane foam shows higher filling ability and discharging ability when a cosmetic composition is supported thereon, and thus is suitable for impregnation with a cosmetic composition. Therefore, the present disclosure provides a dry polyether-based urethane foam having unique properties required for a material for impregnation with a cosmetic composition, not a cosmetic applicator.

According to an embodiment of the present disclosure, the dry polyether-based urethane foam may have a hardness of at least 5, at least 10, at least 15, at least 20, at least 25, at least 30, at least 30, at least 35, at least 35, at least 40 or at least 45, as determined by ASKER Durometer Type F. According to another embodiment of the present disclosure, the dry polyether-based urethane foam may have a hardness of at most 80, at most 75, at most 70, at most 65, at most 60, at most 60, at most 55, at most 50 or at most 45, as determined by ASKER Durometer Type F. According to another embodiment of the present disclosure, the dry polyether-based urethane foam may have a hardness of 5-80, particularly 10-70, more particularly 30-60, and even more particularly 35-55, as determined by ASKER Durometer Type F. The above range of hardness of dry polyether-based urethane foam means the hardness of dry polyether-based urethane foam before it is impregnated with a cosmetic composition. When the dry polyether-based urethane foam has the above-defined range of hardness before it is impregnated with a cosmetic composition, it shows excellent durability and is highly effective in terms of filling ability and discharging ability.

The dry polyether-based urethane foam according to the present disclosure may have a pore number of at least 70, at least 75, at least 80, at least 85, at least 88 or at least 90 per inch (i.e., ppi (pore per inch)). According to another embodiment of the present disclosure, the dry polyether-based urethane foam according to the present disclosure may have a pore number of at most 110, at most 108, at most 105, at most 103 or at most 100 per inch. According to still another embodiment of the present disclosure, the dry polyether-based urethane foam according to the present disclosure may have a pore number of 70-110, particularly 80-105, and more particularly 85-103 per inch. When the urethane foam has a pore number less than 70 ppi, it shows low elasticity, provides no comfort in use, and has difficulty in controlling the flowability of a cosmetic composition. When the urethane foam has a pore number larger than 110 ppi, it shows low durability and provides an unsatisfactory feeling in use of the impregnated cosmetic composition.

According to an embodiment of the present disclosure, the urethane foam may have a reticulated structure. In this case, the urethane foam may be impregnated uniformly with a cosmetic composition with ease at a higher impregnation ratio.

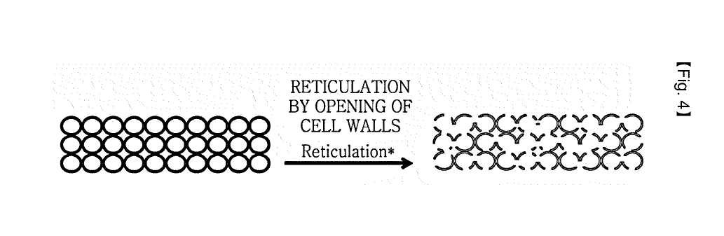

In this context, the urethane foam having a reticulated structure is obtained by subjecting a urethane foam itself to a separate reticulation process. A reticulated structure is also referred to as a three-dimensional network structure. A reticulated structure has a modified network structure obtained by reticulation of the membranes (walls) of balloon-like cells after foaming, and allows not only air permeation but also absorption/discharge of liquid phases. ‘Reticulation’ is a process in which a foam structure is modified into a network structure depending on use. In the case of a sponge foam, this refers to reticulation of air cells through the opening of the walls thereof. Such a reticulation process is shown in FIG. 4. Therefore, the reticulated structure according to an embodiment of the present disclosure is completely different from the conventional foam structure in which three-dimensional membrane structures are formed inside of sponge so that a cosmetic agent may not infiltrate into the deep internal portion of sponge. The reticulated structure is also different from ‘covering’ of the top of a urethane foam support with a net, cloth, non-woven web, etc.

According to another embodiment, the urethane foam may have an open cell structure.

In general, a urethane foam obtained by foaming and aging has a closed cell or semi-open cell structure. When a urethane foam has a closed cell structure, air cells are locked in urethane, and thus it is not easy to impregnate the foam with a low-viscosity emulsified cosmetic composition. Therefore, a urethane foam having an open-cell structure is preferred. According to a particular embodiment of the present disclosure, a urethane foam may have a complete open-cell structure through the above-mentioned reticulation process by which cell surfaces are opened to leave frames only.

The dry polyether-based urethane foam having a reticulated structure according to the present disclosure has lower softness as compared to a wet urethane foam. Due to the characteristics of a reticulated structure, the dry polyether-based urethane foam having an open-cell structure in which the cell surfaces are opened may have low softness. However, since the foam is not to be in direct contact with the skin, it is not necessary for the foam to have high softness. The dry polyether-based urethane foam having such a reticulated structure has higher filling ability and discharging ability, and thus is suitable for a material for impregnation with a cosmetic composition.

As used herein, ‘pore number’ refers to the number of pores per inch, and may be a value obtained by measuring and averaging the numbers of pores present on a 1-inch line based on WI-QA-14 (ASTM standards).

As a ppi number increases, pore size decreases. Since such a pore number is determined by visual inspection, recognition of pores depends on condition of pores, approved in different manners by different countries. For example, Visual Counting Methods (ppi check) of Japan, USA and Europe are shown in the following Table 1.

TABLE 1
Visual Counting Methods (ppi check)

| Determination of | | | |
|---|---|---|---|
| ppi* | Japan | USA | Europe |
| Method | Count only | Count full cells | Count all |
| | full cells | (completely | cells (all cells |
| | (completely | shaped | observed |
| | shaped cells) | cells within the | within the |
| | corresponding | designated | designated |
| | to the | line and on | line and on |
| | designated line | the boundary) | the boundary, |
| | as observed by | observed by a | regardless |
| | a microscope | microscope; | of cell shapes) |
| | | 1.8 times | observed by |
| | | of the value | a microscope; |
| | | determined | 2 times |
| | | in Japan | of the value |
| | | | determined |
| | | | in Japan |

Herein, Visual Counting Method of USA is used to determine ppi. Therefore, the ppi of urethane foam according to an aspect of the present disclosure is different by about 30 ppi from the ppi thereof determined by using Visual Counting Method of Japan.

Herein, cell size is the average of measurements determined by an optical microscope (NIKON ECLIPSE 80i).

According to an embodiment, the dry polyether-based urethane foam may have a density of at least 1, at least 1.3, at least 1.5 or at least 1.8 pcf (pounds per cubic feet). According to another embodiment, the dry polyether-based urethane foam may have a density of at most 3, at most 2.8, at most 2.5, at most 2.3 or at most 2 pcf. According to still another embodiment, the dry polyether-based urethane foam may have a density of 1-3 pcf (pounds per cubic feet), particularly 1-2 pcf. When urethane foam has a density less than 1 pcf, an excessively large amount of cosmetic composition may be discharged, thereby interrupting comfort in use. On the other hand, when urethane foam has a density more than 3 pcf, pores to which a cosmetic composition may be incorporated are insufficient so that effective impregnation with a cosmetic composition may not be accomplished. Herein, density may be determined, for example, based on ASTM D3547.

According to an embodiment, a dry polyether-based urethane foam having a diameter of at least 4 cm, at least 4.2 cm, at least 4.4 cm or at least 4.6 cm and at most 5 cm, at most 4.9 cm or at most 4.8 cm, and a thickness of at least 0.5 cm, at least 0.6 cm or at least 0.8 cm and at most 2 cm, at most 1.8 cm, at most 1.5 cm, at most 1.3 cm or at most 1 cm may allow filling of at least 10 g, at least 13 g or at least 15 g and at most 20 g, at most 18 g or at most 16 g of a cosmetic composition having a viscosity of 5,000-15,000 cps within a time of at least 3 seconds or at least 4 seconds and at most 10 seconds, at most 8 seconds or at most 6 seconds. According to another embodiment, when a dry polyether-based urethane foam having a diameter of at least 4 cm, at least 4.2 cm, at least 4.4 cm or at least 4.6 cm and at most 5 cm, at most 4.8 cm or at most 4.8 cm, and a thickness of at least 0.5 cm, at least 0.6 cm or at least 0.8 cm and at most 2 cm, at most 1.8 cm, at most 1.5 cm, at most 1.3 cm or at most 1 cm supports a cosmetic composition having a viscosity of 5,000-15,000 cps, it is possible to discharge a cosmetic composition in a unit amount of at least 0.2 g, at least 0.3 g or at least 0.4 g and at most 0.7 g, at most 0.6 g or at most 0.5 g.

According to still another embodiment, the cosmetic composition applicable to the dry polyether-based urethane foam for impregnation may be a liquid cosmetic composition, particularly an emulsified cosmetic composition, and more particularly a W/O (water in oil) type or O/W (oil in water) type emulsified cosmetic composition.

According to still another embodiment, an emulsified cosmetic composition may have a viscosity of 5,000-15,000 cps (centipoise), particularly 7,000-13,000 cps, more particularly 8,000-12,000 cps, and even more particularly of 9,000-11,000 cps. When an emulsified cosmetic composition has a viscosity within the above-defined range, it is possible to realize desired effects according to the present disclosure, and the dry polyether-based urethane foam may provide excellent properties. In addition, it is suitable for an emulsified cosmetic composition to have a viscosity within the above-defined range in view of cost-efficiency. According to still another embodiment, the viscosity may be determined by a viscometer, for example, LVDV-II+PRO or RVDV III ULTRA set to spindle No. 63 or spindle No. 64 and a spindle speed of 5 rpm or 12 rpm, but is not limited thereto. The viscosity value may vary with systems used for measurement, spindle number, rpm, or the like.

In another aspect, the present disclosure provides a cosmetic including a dry polyether-based urethane foam impregnated with a cosmetic composition. Since the cosmetic includes the dry polyether-based urethane foam to which a cosmetic composition is applied according to an embodiment of the present disclosure, it is possible to realize high filling ability of cosmetic composition, to discharge an adequate amount of cosmetic composition upon use, and to maintain high durability for a long time. According to an embodiment of the present disclosure, the cosmetic composition may include a cosmetic composition for skin care and a cosmetic composition for make-up. Particularly, the cosmetic composition may be formulated into make-up primer, make-up base, foundation, powder, twin cake, lipstick, lip gloss, eye shadow, eye brow, concealer, lip liner, blusher, UV protector, lotion, cream, or essence, and more particularly, make-up primer, make-up base, liquid or solid foundation, powder, twin cake, lipstick, lip gloss, eye shadow, eye brow, concealer or blusher, but is not limited thereto. According to another embodiment of the present disclosure, the cosmetic may be provided as a cosmetic container generally called ‘pact’ in brief and including a container that has a bottom portion in which the polyether-based urethane foam is received, and a top portion as a lid to which a mirror or the like may be attached. According to still another embodiment of the present disclosure, when a cosmetic including a dry polyether-based urethane foam impregnated with a UV protector is provided in the form of pact, it is possible to use and carry the pact more conveniently as compared to the other UV protector cosmetics and to provide a cooling effect.

The examples, comparative examples, preparation examples and test examples will now be described to describe the construction and effects of the present disclosure in more detail. The following examples, comparative examples, preparation examples and test examples are for illustrative purposes only and not intended to limit the scope of the present disclosure.

### EXAMPLES AND COMPARATIVE EXAMPLES

A dry polyether-based urethane foam and wet polyether-based urethane foam obtained by the method generally known to those skilled in the art are provided. The properties of each type of urethane foam are shown in the following Table 2. The densities in Table 2 are measured based on ASTM D3547.

TABLE 2

| | Dry polyether- | Wet polyether-based |
|---|---|---|
| | based urethane | urethane foam |
| | foam (Example) | (Comparative Example) |
| Structure | Reticulated foam | Reticulated foam |
| Pore number per | 88-100 ppi | 110 ppi |
| inch | | |
| Density (pcf) | 1.8-2.0 | 1.8-2.0 |
| Properties | Pore size is adjustable | Soft |
| | highly elastic | |

### Test Example 1

### Evaluation of Durability

The dry polyether-based urethane foam of Example and the wet polyether-based urethane foam of Comparative Example are evaluated for durability. The test method will be described in detail hereinafter.

Each type of urethane foam (Example and Comparative Example) is formed into a circular shape having a size of diameter 48 mm×thickness 10 mm, and is impregnated with 15 g of the same cosmetic composition having a viscosity of 10,000 cps. Then, each type of foam is stored at 55° C. for 7 days and checked for its condition. The results are shown in the following Table 3. The viscosity may be determined by a viscometer, for example, LVDV-II+PRO or RVDV III ULTRA set to spindle No. 63 or spindle No. 64 and a spindle speed of 5 rpm or 12 rpm.

As can be seen from Table 3, the dry polyether-based urethane foam maintains its shape even after being impregnated with a cosmetic composition, and thus shows high durability.

### Test Example 2

### Evaluation of Filling Ability

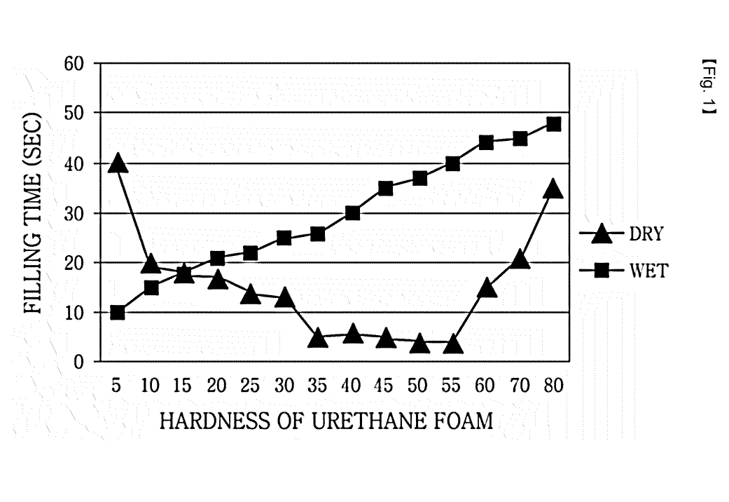

The dry polyether-based urethane foam and the wet polyether-based urethane foam are evaluated for filling ability. Particularly, the hardness of each type of foam is measured by ASKER Durometer Type F before it is impregnated with a cosmetic composition. Then, each type of foam is formed into a circular shape having a size of diameter 48 mm×thickness 10 mm, and the time required for each type of foam to be impregnated with 15 g of the same cosmetic composition having a viscosity of 10,000 cps is determined. The viscosity of cosmetic composition is determined by LVDV-II+PRO set to spindle No. 63 and a spindle speed of 5 rpm. The results are shown in the following Table 4 and FIG. 1.

TABLE 4

| Hardness | Filling time (sec) | |
|---|---|---|
| of | Dry polyether-based | Wet polyether-based |
| foam | urethane foam | urethane foam |
| 5 | 40 | 10 |
| 10 | 20 | 15 |
| 15 | 18 | 18 |
| 20 | 17 | 21 |
| 25 | 14 | 22 |
| 30 | 13 | 25 |
| 35 | 5 | 26 |
| 40 | 6 | 30 |
| 45 | 5 | 35 |
| 50 | 4 | 37 |
| 55 | 4 | 40 |
| 60 | 15 | 44 |
| 70 | 21 | 45 |
| 80 | 35 | 48 |

As can be seen from the above results, when the dry polyether-based urethane foam shows a hardness of 30-55 by ASKER Durometer Type F, the cosmetic composition requires a filling time as short as 10 seconds or less. On the contrary, the wet polyether-based urethane foam requires a longer filling time as the hardness increases, suggesting that it shows low filling ability.

### Test Example 3

### Evaluation of Discharging Ability

(1) Investigation of Optimum Discharge Amount

First, to investigate the amount of a cosmetic composition suitable as a unit discharge amount, 50 persons are allowed to apply different amounts of cosmetic composition and to evaluate each of the following items from which the optimum amount may be derived by rating it as grade 1 to grade 9 (higher grade represents better quality). The results are shown in the following Table 5. “Applicability” refers to a degree of goodness of skin application amount, “cosmetic effect” refers whether or not the cosmetic composition shows excellent coverage on skin without agglomeration and allows uniform make-up within an adequate time, “comfort” refers to a degree of comfort in use of cosmetic composition without take-up many times while allowing easy control of application amount, and ‘satisfaction” refers to a degree of overall satisfaction.

TABLE 5

| Unit | | | | |
|---|---|---|---|---|
| application | | Cosmetic | | |
| amount (g) | Applicability | effect | Comfort | Satisfaction |
| 0.05 | 1 | 1 | 1 | 1 |
| 0.1 | 1 | 1 | 1 | 1 |
| 0.3 | 7 | 5 | 5 | 5 |
| 0.5 | 9 | 9 | 9 | 9 |
| 0.7 | 5 | 5 | 3 | 3 |
| 0.9 | 1 | 3 | 1 | 1 |
| 1.1 | 1 | 1 | 1 | 1 |
| 1.3 | 1 | 1 | 1 | 1 |
| 1.5 | 1 | 1 | 1 | 1 |

As can be seen from the above results, when the unit application amount of a cosmetic composition is 0.3-0.5 g, particularly is about 0.5 g, a high cosmetic effect, comfort and satisfaction are obtained. Therefore, it can be seen that when a cosmetic composition is taken once, 0.3-0.5 g, particularly about 0.5 g is adequate as a unit application amount.

(2) Evaluation of Discharging Ability

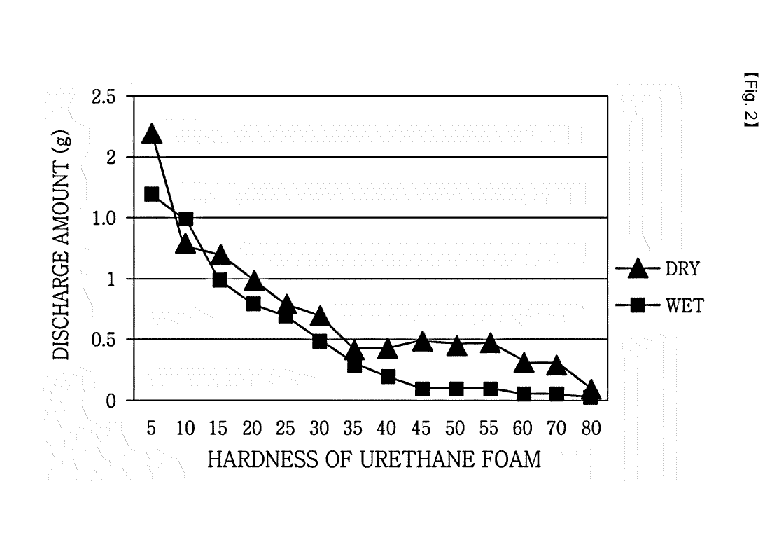

The dry polyether-based urethane foam and the wet polyether-based urethane foam are evaluated for discharging ability. Particularly, the hardness of each type of foam is measured by ASKER Durometer Type F before it is impregnated with a cosmetic composition. Then, each type of foam is impregnated with a sufficient amount of the same cosmetic composition having a viscosity of 10,000 cps, and the amount (g) of cosmetic composition discharged when taking the cosmetic composition once by using puff. The results are shown in the following Table 6 and FIG. 2.

TABLE 6

| Hardness | Discharge amount (g) | |
|---|---|---|
| of | Dry polyether-based | Wet polyether-based |
| foam | urethane foam | urethane foam |
| 5 | 2.2 | 1.7 |
| 10 | 1.3 | 1.5 |
| 15 | 1.2 | 1 |
| 20 | 1 | 0.8 |
| 25 | 0.8 | 0.7 |
| 30 | 0.7 | 0.5 |
| 35 | 0.43 | 0.3 |
| 40 | 0.44 | 0.2 |
| 45 | 0.5 | 0.1 |
| 50 | 0.46 | 0.1 |
| 55 | 0.48 | 0.1 |
| 60 | 0.33 | 0.05 |
| 70 | 0.3 | 0.05 |
| 80 | 0.1 | 0.03 |

As can be seen from the above results, both the dry polyether-based urethane foam and wet polyether-based urethane foam tend to show a drop in amount of cosmetic composition discharged to puff, as the hardness increases. When the dry polyether-based urethane foam has a hardness of 30-60, particularly 35-50 as measured by ASKER Durometer Type F, it shows a unit discharge amount of 0.3-0.5 g, resulting in excellent discharging ability. On the contrary, when the wet polyether-based urethane foam has a hardness of 35 or higher as measured by ASKER Durometer Type F, the unit discharge amount decreases significantly, resulting in poor discharging ability.

In brief, as can be seen from all of the above results, the dry polyether-based urethane foam and the wet polyether-based urethane foam have the durability, filling ability and discharging ability as shown in the following Table 7.

TABLE 7

| | Dry polyether-based | Wet polyether-based |
|---|---|---|
| | urethane foam | urethane foam |
| Filling ability | ⊚ | ◯ |
| Discharging ability | ⊚ | Δ |
| Durability | ⊚ | ⊚ |

As can be seen from the foregoing, the dry polyether-based urethane foam having excellent durability, filling ability and discharging ability of a cosmetic composition is suitable for use in impregnation with a cosmetic composition.

1. A dry polyether-based urethane foam impregnated with a cosmetic composition.

2. The dry polyether-based urethane foam according to claim 1, which has a hardness of 10-70 as measured by ASKER Durometer Type F before being impregnated with a cosmetic composition.

3. The dry polyether-based urethane foam according to claim 1, which has a reticulated structure.

4. The dry polyether-based urethane foam according to claim 1, which has a pore number of 70-110 per inch.

5. The dry polyether-based urethane foam according to claim 1, wherein the cosmetic composition has a viscosity of 5,000-15,000 cps (centipoise).

6. A cosmetic comprising a dry polyether-based urethane foam impregnated with a cosmetic composition.
